(19) United States
(12) Patent Application Publication
(10) Pub. No.: US 2023/0355773 A1
(43) Pub. Date: Nov. 9, 2023

(54) MACROPHAGE TARGETING DRUG CONJUGATES

(71) Applicant: P.I.F. Entrepreneurs Ltd., Jerusalem (IL)

(72) Inventors: Alec M. GOLDBERG, Jerusalem (IL); Samuel H. GOLDBERG, Jerusalem (IL); James I. GOLDBERG, Jerusalem (IL); Isaiah Z. GOLDBERG, Jerusalem (IL); Michael M. GOLDBERG, Jerusalem (IL)

(73) Assignee: P.I.F. Entrepreneurs Ltd., Jerusalem (IL)

(21) Appl. No.: 18/041,134

(22) PCT Filed: Aug. 9, 2021
(86) PCT No.: PCT/IL2021/050968

§ 371 Date: Feb. 9, 2023

Related U.S. Application Data
(60) Provisional application No. 63/158,892, filed on Mar. 10, 2021.
(60) Provisional application No. 63/063,486, filed on Aug. 10, 2020.

(51) Int. Cl. A61K 31/566; A61K 47/54

(52) U.S. Cl. CPC A61K 47/549; A61K 31/566

(57) ABSTRACT

Described herein are novel, macrophage targeting drug conjugates. The macrophage targeting drug conjugates comprise a drug moiety, a mannose moiety, and a linker connecting the drug moiety and the mannose moiety. The linker may comprise a hydrazone group or an oxime group.

### CROSS REFERENCE TO RELATED APPLICATIONS

Benefit is claimed to U.S. Provisional Pat. Application 63/063,486 filed Aug. 10, 2020; and to U.S Provisional Pat. Application 63/158,892 filed Mar. 10, 2021; the contents of which are incorporated by reference herein in its entirety.

### FIELD

Provided herein are novel pharmaceutical agents which can target activated macrophages, and methods of treatment comprising using said pharmaceutical agents.

### BACKGROUND

Macrophages are white blood cells that are involved in the functioning of the immune system. Macrophages are involved in defending the body from pathogens, wound healing, and immune regulation. There are two main phenotypes of macrophages: M1 macrophages, also known as “classically activated” macrophages and M2 macrophages. M1 macrophages are typically pro-inflammatory, while M2 macrophages are typically anti-inflammatory. Various agents can cause activation of macrophages such as cytokines like interferon-gamma, and bacterial endotoxins, such as lipopolysaccharide.

While M1 macrophages are useful for protecting from pathogens, they are also associated with various disease states, in particular, disease states in which inflammation is present.

Thus, a continuing need exists for drugs that can target M1 macrophages, and thereby decrease inflammation and related diseases.

### SUMMARY

Described herein are novel, macrophage targeting drug conjugates. The macrophage targeting drug conjugates comprise a drug moiety, a mannose moiety, and a linker connecting the drug moiety and the mannose moiety. The linker may comprise a hydrazone group or an oxime group.

Without being bound by theory, it is suggested that the mannose moiety may target macrophages by binding a mannose receptor specifically expressed on the surface of an activated macrophage, so that the drug moiety may selectively act on activated macrophages. Due to the rapid internalization of the mannose receptor, a macrophage targeting drug conjugate that binds to the mannose receptor may be internalized into the activated macrophage. The linker, which is optionally a pH sensitive linker, may undergo hydrolysis once internalized by the activated macrophage. In the case of a hydrazone linker, the hydrolysis of the hydrazone group converts it to the corresponding carbonyl group, allowing the drug moiety to act on the macrophage.

It is suggested that drug-conjugates described herein may be administered in lower doses than their corresponding drugs when administered without conjugation to the mannose moiety, due to their specificity and their ability to bind the mannose receptor and target activated macrophages.

### BRIEF DESCRIPTION OF FIGURES

FIG. 1 is a bar graph showing results of an activated macrophage killing assay using various doses of conjugates described herein in contact with activated macrophages;

FIG. 2 is a series of micrographs showing killing of an activated macrophage when contacted with a conjugate described herein;

FIG. 3 is a line graph showing blood glucose elevation in mice administered dexamethasone (solid line) when compared to mice administered equivalent amounts (dashed line) or 25-fold equivalent amount (dotted line) of conjugate described herein;

FIG. 4 is a line graph showing concentration of a macrophage targeting drug conjugate (Compound I5, triangles) and of a corresponding non-conjugated drug (dexamethasone, circles) in plasma of mice over time after administration; and

FIG. 5 is a line graph showing concentration of a macrophage targeting drug conjugate (Compound I5, triangles) and of a corresponding non-conjugated drug (dexamethasone, circles) in urine of mice over time after administration.

### DETAILED DESCRIPTION

### I. Terms

Unless otherwise noted, technical terms are used according to conventional usage. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

### II. Overview of Several Embodiments

Described herein are macrophage targeting drug conjugates (also referred to herein as a “conjugate” or the “conjugates”) which combine a drug moiety and a mannose targeting moiety via a linker, the linker comprising a hydrazone moiety.

The compound according to general formula [I] is an embodiment of such a conjugate.

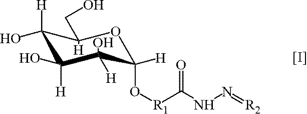

wherein R₁ is a direct bond between the mannose moiety and the carbonyl moiety; or C1-C12 straight alkyl or branched alkyl; and R₂ is a drug moiety.

### Drug:

Optionally, the drug moiety is a steroid. Optionally, the drug is a glucocorticoid or a mineralocorticoid.

The drug moiety may comprise an active portion of a drug. Optionally, an atom of the drug or a portion of the drug may be substituted in order to bind the drug moiety to the linker. For example, a carbon atom of a steroid may be bound to an oxygen atom in the case of a drug. Upon formation of a conjugate, the drug moiety may comprise the carbon atom bound to a nitrogen atom in place of the oxygen atom. The carbon atom and nitrogen atom then form a hydrazone linker. Optionally, the drug moiety has a nitrogen atom at the carbon atom in the 3 position or in the 20 position of the steroid. Optionally, the conjugate acts as a pro-drug of the drug, in that upon administration to the human body, a reaction occurs in which the conjugate is metabolized to form the drug or an active metabolite thereof within the human body.

Optionally, the steroid is a corticosteroid. The corticosteroid is preferably prednisone or dexamethasone. The structures of these corticosteroids are shown below:

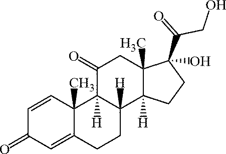

Prednisone is shown.

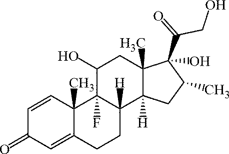

Dexamethasone is shown.

Additional corticosteroids which may be used in the conjugates are selected from the group consisting of: betamethasone, prednisolone, triamcinolone, hydrocortisone, fludrocortisone, and methylprednisolone.

### Linker:

A linker may be a covalent bond. The linker may comprise a carbonyl moiety. Optionally, the linker may comprise a hydrazone moiety, an oxime moiety or an imine moiety. Optionally, the linker may comprise a thioether group. Optionally, the linker comprises a group that undergoes hydrolysis in acidic conditions, optionally, at a pH of 5 or less. The linker may also comprise a C1-C12 alkyl group. The alkyl group may be straight or branched. The linker may also comprise a carbonyl group adjacent to the hydrazone, oxime or imine moiety.

### Mannose:

Mannose is a sugar monomer having the structure C₆H₁₂O₆. Mannose undergoes rapid isomerization among a number of forms, but is primarily present in an α-D-Mannopyranose formation. According to an embodiment, the mannose is bound to a linker or to a drug at the exocyclic oxygen at C1 of the mannose ring.

### Conjugates

Conjugates described herein generally have a structure: M-L-D wherein M is a mannose moiety, L is a linker moiety and D is a drug moiety.

In one embodiment, the conjugate is a dexamethasone conjugate, having the general formula 1D:

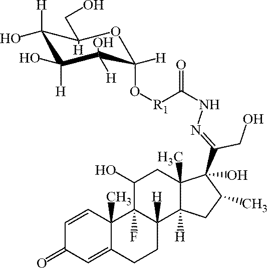

wherein R₁ is a direct bond between the mannose moiety and the carbonyl moiety; or C1-C12 straight alkyl or branched alkyl.

In one embodiment, the conjugate is a prednisone conjugate, having the general formula 1P:

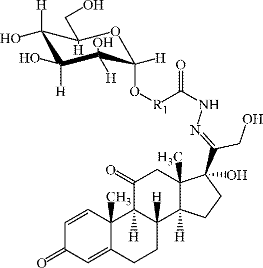

wherein R₁ is a direct bond between the mannose moiety and the carbonyl moiety; or C1-C12 straight alkyl or branched alkyl.

According to an embodiment, the compound is a dexamethasone conjugate designated MD:

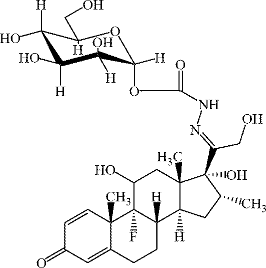

According to an embodiment, the compound is a prednisone conjugate designated MP:

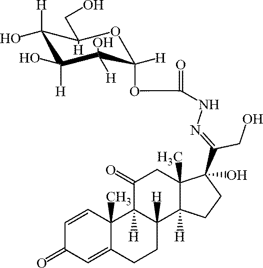

According to an embodiment, the compound is a dexamethasone conjugate designated MD4:

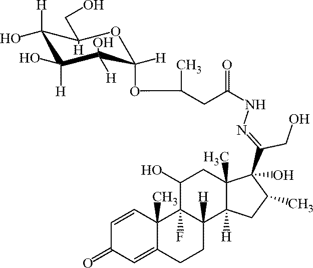

According to an embodiment, the compound is a dexamethasone conjugate designated MD6:

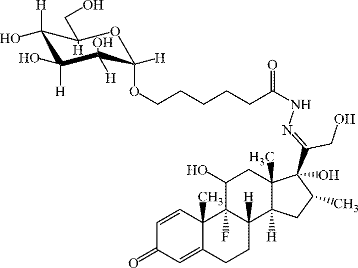

In one embodiment, the conjugate is a prednisone conjugate designated MP.

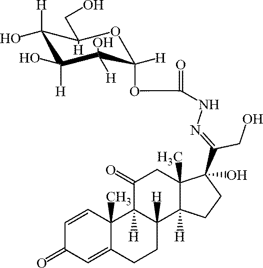

Further embodiments of macrophage targeting drug conjugates comprise one drug moiety per conjugate, and multiple mannose moieties present in each conjugate. The conjugates may each contain between 2 and 10 mannose moieties per conjugate. Preferably, the conjugates each comprise 2, 3 or 4 mannose moieties per conjugate.

Optionally the conjugate is a dexamethasone imine conjugate known as I5 and having the structure designated below:

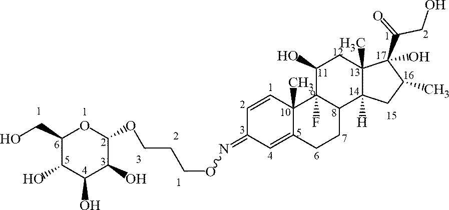

### Methods for Treatment:

Further described herein are methods for treatment of diseases comprising administering to a patient in need thereof, a therapeutically effective amount of a macrophage targeting drug conjugate. Further described herein are macrophage targeting drug conjugates, and pharmaceutical compositions thereof, for treatment of diseases.

The diseases which may be treated using macrophage targeting drug conjugates are diseases which are associated with macrophages. Optionally, the macrophage is an M1 macrophage. Optionally, the disease is an infection disease, an autoimmune disease or an inflammatory disease. The inflammatory disease may be a neuroinflammatory disease, as macrophage targeting drug conjugates have been shown to cross the blood-brain barrier.

Optionally, the drug may act to convert M1 macrophage to M2 macrophages. This is particularly relevant to autoimmune disease.

Optionally, the diseases which may be treated using macrophage targeting drug conjugates are diseases associated with targeting an M2 macrophage. The disease may be a fibrotic disease or a disease associated with a tumor.

Optionally, the autoimmune disease to be treated is selected from the group consisting of: rheumatoid arthritis, autoimmune enteropathy, psoriasis, dermatitis, alopecia, immunodysregulation polyendocrinopathy enteropathy X-linked syndrome and autoimmune endocrinopathies.

Optionally, the disease is non-alcoholic fatty liver disease or non-alcoholic steatohepatitis.

Optionally the disease is a neuroinflammatory disease. Optionally, the disease is selected from the group consisting of: multiple sclerosis, neuromyelitis optica, optic neuritis, Alzheimer’s disease and transverse myelitis.

The diseases which may be treated using macrophage targeting drug conjugates may include Parkinson’s disease.

The diseases which may be treated using macrophage targeting drug conjugates, a lipid storage disease. Optionally, the lipid storage disease is selected from the group consisting of: Gaucher’s disease, Niemann-Pick disease, Fabry’s disease, Farber’s disease and Tay-Sachs.

The diseases which may be treated using macrophage targeting drug conjugates may include asthma.

The diseases which may be treated using macrophage targeting drug conjugates may include depression, drug addiction, and opioid addiction.

The diseases which may be treated using macrophage targeting drug conjugates may include cardiovascular disease, including but not limited to atherosclerosis.

Additional diseases which may be treated with macrophage targeting drug conjugates may include viral diseases or protozoan diseases. Optionally, the viral disease is resultant from a coronavirus infection. Optionally, the disease is COVID-19. Optionally, the disease is post viral syndromes including complications resulting from prior infection with, MERS, SARS, COVID-19 (long COVID), and non-coronavirus pathogens like Ebola. Optionally, the disease is leishmaniasis. Optionally, the disease is an antibiotic resistant strain of staphylococcus, streptococcus, E.coli or other infectious pathogens.

Without being bound by theory, it is suggested that treatment of viral disease such as coronavirus is associated with macrophages, which serve as a first line of defense in the organism in which a virus is present. The virus is internalized by the macrophage and the macrophage begins to eliminate the virus by coordinating the innate and adaptive immune responses to eliminate the virus. The macrophages release a variety of chemokines and cytokines along with expressing certain receptors including CD 206. The organism infected by the virus may be relatively symptom free at this early stage of viral infection. The virus may then take control of the macrophage and use it to enable viral replication. At this stage, a macrophage may be targeted with a macrophage targeting drug conjugate comprising an active ingredient such as a glucocorticoid to provide efficient delivery of the glucocorticoid to the macrophage, thereby initiating apoptosis in the cell, thereby inactivating the virus. Optionally, the macrophage targeting drug conjugate may be administered at a later stage, once a patient is exhibiting symptoms, for example of the respiratory system, associated with excess immune activity. The immune activity may be modulated by killing macrophages using macrophage targeting drug conjugates.

Coronaviruses (CoVs) are members of the order Nidovirales, which includes enveloped viruses with large (~30 kb), positive-sense single-stranded RNA genomes that yield a characteristic nested set of subgenomic mRNAs during replication in the cytoplasm of infected cells. The genome organization for coronaviruses is highly conserved, with the 5′-most two-thirds of the genome encoding the replicase polyprotein, followed by sequences encoding the canonical structural proteins: spike, envelope, membrane, and nucleocapsid. Many CoVs contain accessory genes, which are interspersed among the genes for the structural proteins. Although these accessory genes are not necessarily required for virus replication and are, in general, not highly conserved within the virus family, many encode proteins that regulate the host response. Interestingly, coronavirus replicase proteins, which are highly conserved, can also act as antagonists to block or delay the host innate immune response to infection. That a slew of coronavirus-encoded accessory and non-accessory proteins have been shown to shape the host antiviral response suggests that viral-mediated subversion of host defenses is an important element of infection.

When dealing with pandemics those involved in drug development do not have the luxury of time nor the ability to predict the rate of change in the virus nor the frequency of new variants. In the case of the Coronavirus there have been multiple viruses impacting humans including SARS, MERS and SARS-CoV-2. Vaccines vs SARS and MERS if available would need to be modified for SARS-CoV-29 and once developed may or may not provide a benefit for the next Coronavirus to develop or even remain effective for a virus that has spread as widely as SARS-CoV-2 and thus been exposed to so many mutations.

What is beneficial for SARS-CoV-2 and for future similar viruses is a therapeutic that ideally can be dosed before permanent damage occurs in the patient, thus before a massive inflammatory response is generated to combat the massive number of virus infected cells. The therapeutic should target the virus soon after infection but before significant replication can occur. The therapeutic should contact the virus and be effective against a highly conserved viral defense mechanism and not one unique to any viral strain. The viral defense target should be highly conserved and thus not one amenable to mutation. The dose of the therapeutic should be safe and effective regardless of the stage of infection. In other words, the dose may be the same for the patient that was just infected or the patient that has symptoms, as the virus has had the opportunity to vastly expand over days to weeks. Optionally, the dose may be lower for an asymptomatic patient than for a symptomatic patient.

Optionally, the macrophage targeting drug conjugates described herein may be administered to a subject at an early stage of viral infection, or at a late stage. At a late stage, macrophage targeting drug conjugates may be able to limit or prevent cytokine storm associated with viral infection. Cytokine storm is a systemic inflammatory syndromes involving elevated levels of circulating cytokines and immune-cell hyperactivation, and can be life threatening. An advantage of such an antiviral treatment is that the patient will obtain immunity to the virus after being exposed to the virus. Optionally, the macrophage targeting drug conjugates may be administered to early convalescent phase patients.

Acute hyperglycemia is regarded as a risk factor for critically ill patients and has been identified as an independent risk factor for adverse outcomes in such patients such as severe infections, multiple organ failure, and death. Acute hyperglycemia has also been found to cause long term damage to insulin secreting islet cells. Patients with Type II diabetes or metabolic syndrome are especially susceptible to severe COVID-19 after infection. Acute hyperglycemia causes difficulty to control glucose in such patients in an intensive care unit setting.

It is known that glucocorticoids significantly increase blood glucose levels in patients. In the RECOVERY trial in which dexamethasone was administered to COVID patients, blood glucose levels were significantly elevated in diabetics (Rayman G, Lumb AN, Kennon B, Cottrell C, Nagi D, Page E, Voigt D, Courtney HC, Atkins H, Higgins K, Platts J, Dhatariya K, Patel M, Newland-Jones P, Narendran P, Kar P, Burr O, Thomas S, Stewart R. Dexamethasone therapy in COVID-19 patients: implications and guidance for the management of blood glucose in people with and without diabetes. Diabet Med. 2021 Jan;38(1):e14378. doi: 10.1111/dme.14378. Epub 2020 Sep 21). Although dexamethasone may be beneficial in COVID-19, extreme care must be taken when administering to patients in which acute hyperglycemia is especially problematic, such as diabetic patients and patients with metabolic syndrome.

It has been surprisingly found that macrophage-targeting drug conjugates, although they contain glucocorticoids as an active component, are not associated with hyperglycemia.

It has been suggested that glucocorticoids can have immunosuppressive effects. This can be harmful in patients such as COVID-19 patients, as a decrease in anti-viral interferon response may result in decreased viral clearance. Macrophage targeting drug conjugates appear not have immunosuppressive activity as they do not prevent the activation of macrophages nor do they block the activity of cytokines that, in addition to the virus and the damage associated molecular patterns, drive the activation of uninfected macrophages, and only exhibit anti-inflammatory activity resulting from their activation in CD206 expressing macrophages.

In addition, it is suggested that macrophage targeting drug conjugates will show a decrease in M2 activity, thereby potentially decreasing lung fibrosis in COVID-19 patients. It is also suggested that macrophage-targeting drug conjugates do not lower cell T-cell and B-cell number, although glucocorticoids alone have shown to be toxic to T-cells and B-cells. These effects of macrophage targeting drug conjugates mentioned above are supported by the showing that they effectuate different cytokine expression levels in serum, BALF and the CNS as compared to free corticosteroids, such as dexamethasone.

Optionally, macrophage targeting drug conjugates may be administered alone or in combination. An exemplary combination for treatment of virus such as coronavirus is with an antimalarial drug such as hydroxychloroquine. Another exemplary combination for treatment of virus such as coronavirus is with azithromycin.

Macrophage targeting drug conjugates may be administered in a variety of routes. According to an embodiment, they are administered via the oral, nasopharyngeal, subcutaneous, intravenous, intrathecal, intraocular, intra-articular, inhaled, or topical routes.

According to an embodiment, the macrophage targeting drug conjugates are administered in a molar amount which is less than the corresponding drug. For example, a macrophage targeting drug conjugate comprising dexamethasone as a drug moiety, may be administered in a molar amount of between 0.001% to 50% relative to the molar amount indicated for use for a given indication.

According to an embodiment, the macrophage targeting drug conjugates in a molar amount which is more than the corresponding drug. For example, a macrophage targeting drug may be administered in a molar amount of between 150% and 1000% relative to the molar amount indicated for use for a given indications. Without being bound by theory, this type of administration is possible because of the lower toxicity of the macrophage targeting drug conjugates described herein, due to the selectivity of the conjugates and to the understanding that macrophage targeting drug conjugates are active inside microphages, and not active (or are less active) when contacting other cells/ tissues.

Further embodiments relate to pharmaceutical compositions comprising a macrophage targeting drug conjugate. According to an embodiment, a macrophage targeting drug conjugate is combined with at least one pharmaceutically acceptable excipient to form a pharmaceutical composition. In an embodiment, the pharmaceutical composition is adapted for human or animal use via oral, nasopharyngeal, subcutaneous, intravenous, intrathecal, intraocular, intra-articular, inhaled, or topical administration.

The pharmaceutical compositions according to an embodiment may be conveniently presented in unit dosage form and are prepared by any of the methods well known in the art of pharmacy. In an embodiment, the unit dosage form is in the form of a tablet, capsule, lozenge, wafer, patch, ampoule, vial or pre-filled syringe.

The pharmaceutical compositions are generally administered in the form of a pharmaceutical composition comprising at least one active component together with a pharmaceutically acceptable carrier or diluent.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tableting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the active agents can be combined with various sweetening agents, flavoring agents, coloring agents, emulsifying agents and/or suspending agents, as well as such diluents as water, ethanol, propylene glycol, glycerin and various like combinations thereof.

The compositions according to embodiments may also be administered in a controlled release formulation such as a slow release or a fast release formulation. Such controlled release dosage composition may be prepared using methods well known to those skilled in the art.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

Pharmaceutical compositions according to embodiments may contain an amount of 0.1%-95% of the active agent, preferably 1%-70%. In an embodiment, the daily dosage of the active agent is between 0.001 mg and 3000 mg.

Without being bound by theory, it is suggested that macrophage targeting drug conjugates are advantageous relative to the corresponding drugs due to the selectivity of the macrophage targeting drug conjugates. While they may be administered systemically, it is suggested that macrophage targeting drug conjugates will primarily release the active drug moiety in the proximity of, preferably, inside the activated macrophage. This targeted administration limits “off target” toxicity, increases safety, and allows for selective treatment of diseases associated with activated macrophages.

U.S. Pat. Application publication 2018/0099048 discloses dextran-based drug conjugates. Macrophage targeting drug conjugates are advantageous described herein are advantageous relative to conjugates using dextran for a number of reasons. The previously described dextran-based compounds are based on a 10,000 dalton dextran backbone. The starting dextran available and sold as dextran USP is never exactly 10,000 daltons and has a broad range with an average MW of 10,000. After drug conjugation, the conjugate has a molecular weight of close to 20,000 daltons and is highly heterogenous. It has an approximate number of d mannose binding moieties (15-20) and an approximate number of free linker sites (depending in part on number of d mannoses already linked). When designing a therapeutic there will be an approximate number of therapeutic molecules attached to each molecule of backbone. When such a platform is used for an imaging agent that are typically dosed once and at a very low dose in microgram levels per administration the variability is acceptable given the safety. However, for therapeutics dosed at mg levels the variability, stability, and various metabolites would make a dextran-based therapeutically practically extremely difficult to characterize and reproducibly produce under GMP.

Large molecular weight dextran-based products are likely to have immunogenic properties due to their size making repeat dosing problematic and toxicity testing in animals less predictive of human toxicity.

The drug conjugates described herein are simple organic molecules with standard pharmaceutical manufacturing properties having molecular weights of about 600 daltons. Since there is no polymer backbone these compounds can be made with high purity and thereby providing straightforward regulatory approval processes. Such pure compounds are much easier to qualify, thus quicker and less expensive to develop. The drug conjugates describe herein are easier to formulate and have greater systemic bioavailability.

Macromolecules are significantly disadvantaged crossing biological barriers as compared to small molecules. Agents described herein cross the blood-brain barrier, whereas 20kd polymers likely cross at a few orders of magnitude lower efficiency. Furthermore, macromolecules have lower intra tumor penetration than small molecules.

According to an embodiment, provided is a compound having the formula M-L-D wherein M is a mannose moiety, L is a linker moiety and D is a drug moiety, wherein the drug moiety is a corticosteroid. Optionally, the linker comprises a hydrazone moiety, an oxime moiety, an imine moiety, and a thioether moiety. Optionally, L further comprises a carbonyl group. Optionally, the carbonyl group is bound to the hydrazone moiety. Optionally, L is a linker comprising an oxime moiety. Optionally, L further comprises a C1-C12 alkyl group. Optionally, the C1-C12 alkyl group is bound to the carbonyl group. Optionally, the alkyl group is a C1-C6 alkyl group. Optionally, the steroid is selected from the group consisting of: prednisone, dexamethasone, betamethasone, prednisolone, triamcinolone, hydrocortisone, fludrocortisone, and methylprednisolone. Optionally, the drug is prednisone or dexamethasone. Optionally, the compound has a molecular weight of less than 800.

According to an embodiment, provided is a compound according to the formula:

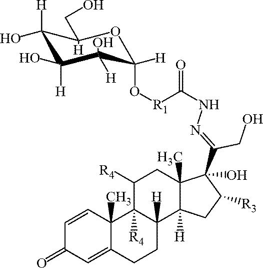

wherein R₁ is a direct bond; or C1-C12 straight alkyl or branched alkyl; R₂ is hydrogen or fluorine; R₃ is a hydrogen or methyl group. R₄ is a hydroxyl group or a ketone group. Optionally, R₁ is a direct bond. Optionally, R₁ is a CH₂ group. Optionally, R₁ is (methyl)ethyl group. Optionally, R₁ is a pentyl group. Optionally, R₂ and R₃ are H and R₄ is a ketone group. Optionally, R₂ is fluorine, R₃ is methyl and R₄ is a hydroxyl group. According to an embodiment, provided is a compound according to the formula:

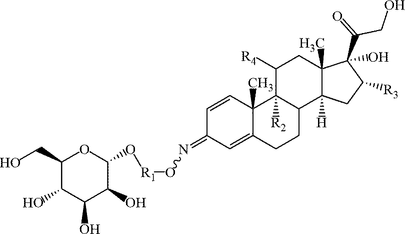

wherein R₁ is a direct bond; or C1-C12 straight alkyl or branched alkyl; R₂ is hydrogen or fluorine; R₃ is a hydrogen or methyl group, and R₄ is a hydroxyl group or a ketone group. Optionally, R₁ is a (CH₂)₃ group. Optionally, R₂ and R₃ are H and R₄ is a ketone group. Optionally, R₂ is fluorine, R₃ is methyl and R₄ is a hydroxyl group.

According to an embodiment, provided is a pharmaceutical composition comprising a compound described in one of the aforementioned embodiments.

According to an embodiment, provided is a method for treatment of a disease comprising administering to a patient in need thereof a compound described in one of the aforementioned embodiments. Optionally, the disease is associated with increased macrophage activation. Optionally, the disease is an autoimmune disease or an inflammatory disease. Optionally, the disease is selected from the group consisting of: rheumatoid arthritis, autoimmune enteropathy, psoriasis, dermatitis, alopecia, immunodysregulation polyendocrinopathy enteropathy X-linked syndrome and autoimmune endocrinopathies. Optionally, the disease is non-alcoholic fatty liver disease or non-alcoholic steatohepatitis. Optionally, the disease is a neuroinflammatory disease. Optionally, the neuroinflammatory disease is selected from the group consisting of: multiple sclerosis, neuromyelitis optica, optic neuritis, Alzheimer’s disease and transverse myelitis. Optionally, the disease is Parkinson’s disease. Optionally, the disease is a lipid storage disease. Optionally, the disease is selected from the group consisting of: Gaucher’s disease, Niemann-Pick disease, Fabry’s disease, Farber’s disease and Tay-Sachs. Optionally, the disease is asthma. Optionally, the disease is selected from the group consisting of: depression, drug addiction, and opioid addiction. Optionally, the disease is selected from the group consisting of: atherosclerosis and cardiovascular disease. Optionally, the disease is a pathogen, comprising a viral disease, a bacterial disease or a protozoan disease. Optionally, the disease is resultant from a coronavirus infection. Optionally, the disease is COVID-19. Optionally, the patient does not have COVID-19 symptoms. Optionally, the disease is leishmaniasis. Optionally, the patient is suffering from cytokine release syndrome. Optionally, the patient is also suffering form a disease associated with a metabolic disorder associated with glucose metabolism. Optionally, the metabolic disorder is diabetes or metabolic syndrome. Optionally, the disease is a disease of the brain, optionally, a neurological disease. Optionally, the disease is a cancer of the brain, optionally, glioblastoma.

According to an embodiment, provided is a method for treatment of a disease by selectively delivering a corticosteroid to an immune cell. Optionally, the immune cell is a macrophage. Optionally, the method comprises administering a compound described above.

### EXAMPLES

### Example 1A

### Preparation of Compounds According to Formula (I)

Compounds according to formula (I) comprising a mannose moiety, a linker having a carbonyl group and a hydrazone moiety, linked to a drug moiety which is a corticosteroid may be prepared according to the following general procedure:

A solution of 1,2,3,4,6-penta-O-acetyl-D-mannopyranose (6 g, 15.37 mmol) and 3-hydroxybutyric acid (2.24 g, 21.5 mmol, 1.4 eq) in DCM (120 mL) at 5° C. is added drop wise to BF₃ .Et₂O (boron trifluoride diethyl etherate 9.5 mL, 77 mmol, 5 eq). The mixture is stirred at 5° C. for 24 h. More acid (0.2 eq) is added, followed by drop-wise addition of BF₃ .Et₂O (4.75 mL). The mixture is stirred at 5° C. for another 6 h. The mixture is quenched by sat. NaHCO₃ (100 mL). The two layers are separated, and the organic layer is extracted with sat. NaHCO₃ (3x100 mL). The combined aqueous layers are acidified with 6N HCl solution and extracted with DCM (3x100 mL). The combined extracts were dried and concentrated to obtain crude product.

A solution of the starting ketone (10 g, 25.51 mmol) in EtOH (600 mL) is added to hydrazine monohydrate (2.55 g, 2 eq). The reaction mixture is heated at 50° C. for 2 days. After cooling to rt, the mixture is concentrated to a volume of ~100 mL. The residue is poured into H₂O (500 mL), and the product is precipitated. The mixture was filtered, and the solid washed with H₂O. The collected solid is dried on vacuum to yield the product which was used in the next step.

A mixture of above crude product (2.95 g, 6.79 mmol) and hydrazone (3.17 g, 7.8 mmol, 1.15 eq) and HATU (3.37 g, 8.87 mmol, 1.3 eq) in THF (40 mL) is added to N,N-Diisopropylethylamine (iPr2NEt) (2.47 mL, 14.16 mmol, 2 eq). The resulting mixture is stirred at rt for 3 h. The mixture is diluted with DCM (150 mL) and washed with 1 N HCl (50 mL), H2O (50 mL) and sat. NaHCO₃(50 mL), successively. The organic layer is dried (Na₂SO₄) and concentrated. The residue is purified by column chromatography to provide crude product.

The crude product from above step is dissolved in 50 mL of MeOH/Et₃N/H2O. The reaction mixture is stirred at rt overnight. LC-MS The mixture is concentrated to dryness, and the residue is purified by column chromatography, followed by reverse-phase column chromatography to obtain the product.

The precursor of the acid and hydrazone compounds is prepared from 1,2,3,4,6-penta-O-acetyl-D-mannopyranose and corresponding acid with hydrazine to yield designed compounds based on the same procedure of the compound above.

### Example 1B: Manufacture of Compound I5

A conjugate comprising dexamethasone, a linker and a mannose moiety was prepared using the following procedure. Other conjugates having alternate linkers and drugs may be manufactured using the same process described in this example, with appropriate modifications for the alternate drug and linker.

### Step 1: (2R,3R,4S,5S,6S)-2-(acetoxymethyl)-6-(3-bromopropoxy)tetrahydro-2H-pyran-3,4,5-triyl triacetate (Compound R1)

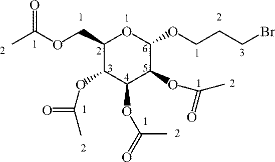 [SMILES: CC(=O)OC[C@H]1O[C@H](OCCCBr)[C@@H](OC(C)=O)[C@@H](OC(C)=O)[C@@H]1OC(C)=O]

1,2,3,4,6-Penta-O-acetyla-D-mannopyranoside (8 gr, 20.5 mmol) was dissolved in CH₂Cl₂ (80 mL) and 3-bromopropan-1-ol (3.13 gr, 22.5 mmol) was added, followed by the addition of boron trifluoride etherate (10.1 mL, 82.0 mmol). The reaction was stirred in the dark under a nitrogen atmosphere for 24 h. TLC analysis (Hexane:EtOAc=1:1) was performed. New spot was detected. DCM was added and the reaction mixture was neutralized by adding saturated NaHCO₃ solution. The phases were separated, the aqueous phase was washed with DCM. The combined organic phase was dried over Na₂SO₄, filtered and evaporated. The crude product was purified by column chromatography (gradient of 3:1 to 1:1 - Hexane:EtOAc), the product was isolated as a colorless oil in a yield of 64%.

### Step 2: (2R,3R,4S,5S,6S)-2-(Acetoxymethyl)-6-(3-((1,3-Dioxoisoindolin-2-yl)oxy)propoxy) Tetrahydro-2H-Pyran-3,4,5-Triyl Triacetate (Compound R2)

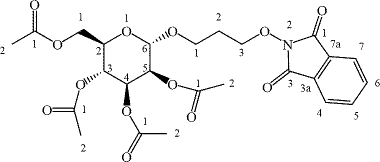 [SMILES: CC(=O)OC[C@H]1O[C@H](OCCCON2C(=O)c3ccccc3C2=O)[C@@H](OC(C)=O)[C@@H](OC(C)=O)[C@@H]1OC(C)=O]

To a solution of compound R1 (3.5 gr, 7.5 mmol) and N-hydroxylphthalimide (1.35 gr, 8.25 mmol) in DMF (15 mL), DBU (1.1 mL, 8.25 mmol) was added and red solution was stirred for 18 h at room temperature under nitrogen atmosphere. The reaction was monitored by TLC (Hexane/EtOAc=1/1) and LCMS (SB1090), no starting material remained. The yellow-orange solution was added dropwise to the solution of 1N HCl (50 mL). White solid was separated, which was dissolved in EtOAC. The aqueous phase was extracted with EtOAc, dried over Na2SO4, filtered and evaporated. The crude product was purified by silica gel chromatography (gradient of 0-50% EtOAc in Hexane). 3 gr (73% yield) of white solid was obtained.

### Step 3: (2R,3R,4S,5S,6S)-2-(Acetoxymethyl)-6-(3-(Aminooxy)propoxy)Tetrahydro-2H-Pyran-3,4,5-Triyl Triacetate (Compound R3)

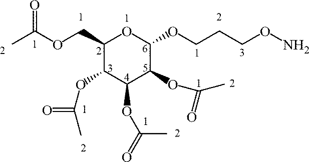 [SMILES: CC(=O)OC[C@H]1O[C@H](OCCCON)[C@@H](OC(C)=O)[C@@H](OC(C)=O)[C@@H]1OC(C)=O]

Compound R2 (3.04 gr, 5.51 mmol) was dissolved in methanol (100 mL) for 30 min due to the hard dissolution and 1.5 eq. of hydrazine hydrate (0.401 mL, 8.27 mmol) was added. The stirring was continued for 4 h, the reaction was monitored by TLC (EtOAc) and LCMS (SB1090). The solvent was removed by evaporation, white solid precipitated (by-product). The crude product was washed with EtOAc, the suspension was filtered, and the mother liquor was evaporated. This washing with EtOAc was repeated 4 times, followed by an additional wash with CHCl₃. The mother liquor was evaporated to dryness. 1.85 gr (80% yield) of colourless oil was obtained. The NMR and LCMS (SB1090) analyses conform the structure.

### Step 4: (2R,3R,4S,5S,6S)- 2- (Acetoxymethyl)- 6-(3-(((8S,9R,10S,11S,13S,14S,16R,17R,E)- 9-Fluoro-11,17-Dihydroxy-17-(2-Hydroxyacetyl)-10,13,16-Trimethyl-6,7,8,9,10,11,12,13,14,15,16,17-Dodecahydro-3H-Cyclopenta[a]phenanthren-3-ylidene)amino)propoxy) Tetrahydro-2H-pyran-3,4,5-triyl Triacetate (Compound R4)

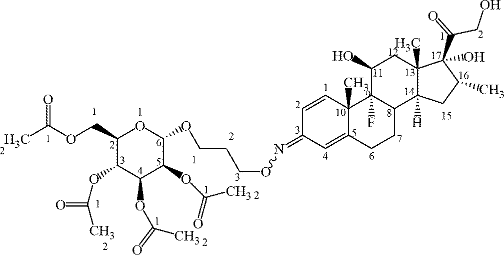

The previously prepared oxime R3 (1.8 gr, 4.27 mmol) was added into a solution of dexamethasone (1.11 gr, 2.85 mmol) in EtOH (25 mL), followed by PTSA (0.27 gr, 1.42 mmol). The reaction mixture was refluxed for 4h while monitoring by LCMS (SB1090). After 1 h, conversion of 95% was detected. Products of mono/di deprotection of acetate groups were also observed by LCMS. The reaction mixture was cooled to room temperature, NaHCO₃ (1 g) was added and the suspension was stirred for 5 min and filtered. EtOH was evaporated to dryness. Purification by column chromatography (gradient 50% to 100% of EtOAc in Hexane) provided a mixture of the product and partially deprotected by-product. Yield: 1.66 gr. This mixture was used in the next step.

### Step 5: 1-((8S,9R,10S,11S,13S,14S,16R,17R,E)-9-Fluoro-11,17-Dihydroxy-10,13,16-Trimethyl-3-((3-(((2S,3S,4S,5S,6R)-3,4,5–Trihydroxy-6-(Hydroxymethyl)Tetrahydro-2H-Pyran-2-yl)oxy)propoxy)imino)-6,7,8,9,10,11,12,13,14,15,16,17-Dodecahydro-3H-cyclopenta[a]phenanthren-17-yl)-2-Hydroxyethan-1-One (Compound I5)

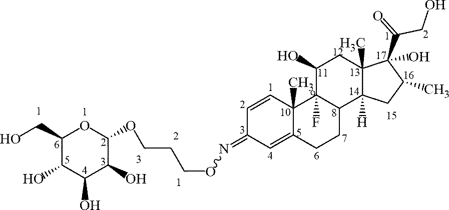

Compound R4 (1.66 gr) was dissolved in 60 mL of MeOH/Et₃N/H₂O (8:1:1). The reaction mixture was stirred at room temperature and monitored by LCMS (SB1090). After 4h of stirring, only the desired product (m/z 628) was observed. The solvent was evaporated and the product (1.33 g of crude product) was purified by silica gel chromatography, eluted with DCM/MeOH=85/15 and monitored by TLC (DCM/MeOH= 80/20). 0.7 gr of white solid was isolated and characterized by NMR and LCMS (SB1090), which confirms the structure.

### Example 2A: Binding of Conjugates to Immobilized CD206 Presenting Cells

Materials used in this example are commercially available. Unless otherwise indicated, the methods follow standard procedures. A Chip assay (chromatin immunoprecipitation) (BiacoreT200) was employed in order to investigate the interaction of various small molecules with immobilized CD206 protein, (CM5 with 5000 resonance units (RU) CD206 immobilized on Fc2).

Immobilization: The CD206 proteins were immobilized as a 50 µg/mL solution in 10 mM sodium acetate, with a pH of 4.5, using standard amine coupling chemistry (EDS/NHS activation). The compounds tested appear in the table.

Running and sample buffer: assay conditions were HBS-P, 0.5 mM, CaCl₂, with a pH of 8.0 (10 mM HEPES, 150 mM NaCl, pH 7.4 with 0.005% Tween 20) at 25° C.

Regeneration buffer: 30 second injections of 0.1 % SDS and of 10 mM NaOH.

Flow and injections scheme: 50 uL/minute, 3-minute injection, 2-4-minute disassociation. A double reference method was used for analysis.

Sample preparation: Samples were prepared as two-fold dilution series with the starting concentration of 700 nM in the running buffer (43.8, 87.5, 175, 350, 700 nM).

### Results

Table 1, below, summarizes the results obtained.

| Material | MW | Purity % | Mg | ml | KD (mM) Exp. 1 | KD (mM) Exp. 2 |
|---|---|---|---|---|---|---|
| MP | 592.2 | 95 | 0.91 | 0.505107 | 1.16 | 0.92 |
| MD4 | 654.2 | 90 | 0.97 | 0.53841 | 0.24 | |
| MD6 | 682 | 98 | 5.36 | 2.975133 | 3.67 | |
| MD | 626.1 | 90 | 0.98 | 0.543961 | 1.13 | |
| I5 | 627.3 | 98 | 1.59 | 0.882549 | | 1.09 |

### Conclusion

All steady state analysis was performed by taking measurements at the binding maximum. Some of the compounds displayed reduction of signal with protracted injection most likely caused by effects of impurities or remaining solvents in the samples. The most pronounced effect was found for the MD compound. These results indicate that the compounds described herein could be promising new therapeutic means for treating diseases mediated by activated macrophages expressing the mannose receptor (CD206).

### Example 2B: Macrophage Assay

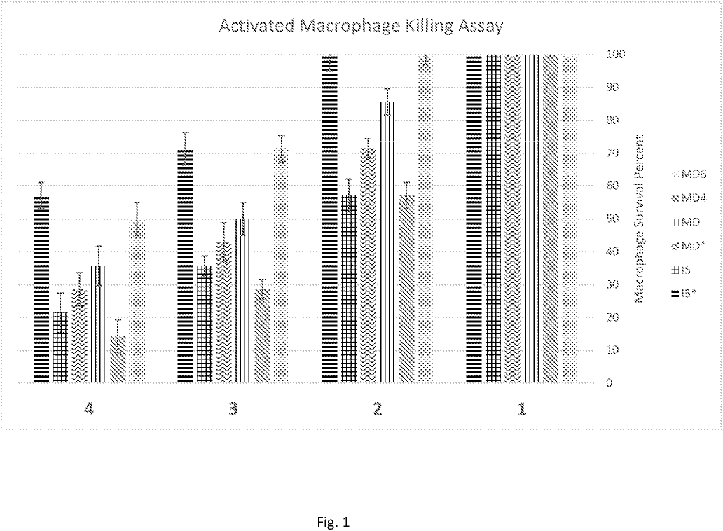

A macrophage killing assay was performed in order to determine potential of conjugates described herein for treatment of macrophage-related diseases. THP1 monocytes were transformed into macrophages for using phorbol myristate acetate (PMA). Macrophages, after activation by infection with Leishmania, were incubated with conjugates described herein at 10, 1 and 0.1 microgram (µg) per milliliter (ml) concentration of compounds. The impact of test compounds on killing activated (infected) macrophages was compared to impact of test compounds killing on macrophages which had not been infected and were inactivated. The data shown in FIG. 1 represents macrophage survival percent after 12-14 hours of incubation with conjugates. The conjugates used herein were MD6, MD4, MD and I5. The asterisks of MD* and I5* represented compounds having various levels of optical purity relative to their counterparts. In the figure, series 1 represents control macrophages which were not infected. Series 2, 3 and 4 represent the 0.1, 1 and 10 µg/ml dosages. The values shown in these series represented number of cells, as normalized to control, non-infected macrophages.

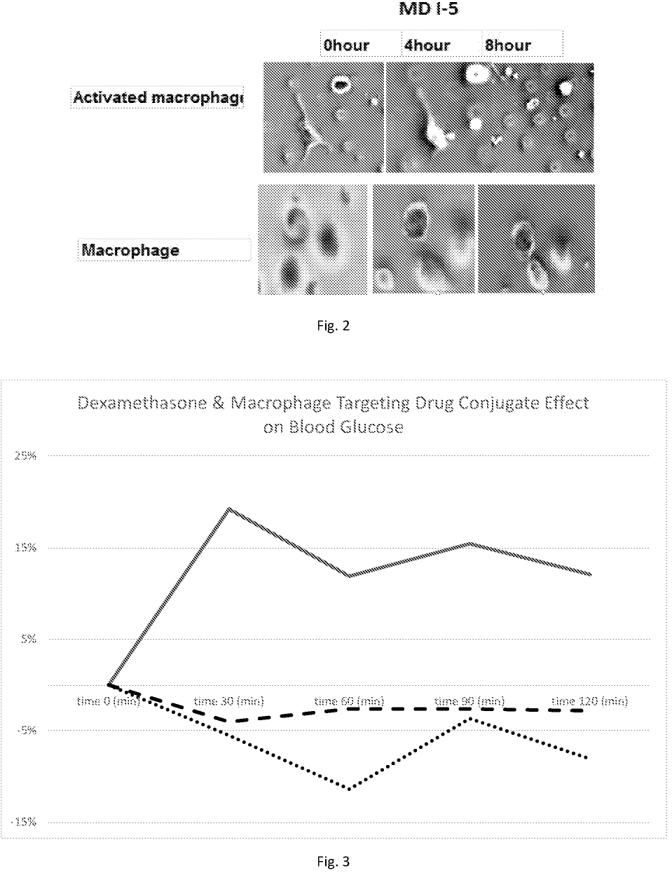

FIG. 2 shows another experiment in which one test conjugate, I5, was added to activated (top row) or non-infected macrophages (bottom row) and was monitored under live imaging, while incubated with I5 at the concentration of 10 µg/ml. Olympus cellsense live imaging was performed with compound I5 at a concentration of 10 µg/ml showed killing of activated macrophages within 8 hours.

Media used for macrophage assay: RPMI with 10% FCS, supplemented with pencillin streptomycin and glutamine. The compounds were prepared in phosphate buffered saline with pH adjusted to 7.6 and were used immediately after preparation.

Infection of macrophage: The macrophages were infected with 5x times with Leishmania donovani - for a density of 1x10^5 monocytes transformed to macrophage, the infection density is 5 x 10^5 L.donovani.

The experiment was performed according to the following schedule:
Monocyte to macrophage transformation: 0 to 48 hours
Macrophage Infection with Leishmania: 48-84 hours
Treatment with compound: 84 - 96 hours
Plate used for assay: Microscopy compatible glass bottom plate -24 wells.

The macrophages that were alive were counted in five different fields of the wells for different concentration and the average count was used for the assay. Based on the count of the control macrophages, the % was adjusted.

Results: FIG. 1 shows that the tested compounds decrease activated macrophage survival in a dose-dependent manner. As dose is increased, macrophage survival decreases for all tested compounds. Conjugates I5 and MD4 were particularly effective. FIG. 2 shows that activated macrophage shown at 0 hours in upper row, with dendrites, was eliminated within 8 hours of contact with conjugate I5. In contrast, macrophages which had not been activated by infection (lower row) remained unchanged in presence of the same concentration of I5. These results indicate the potential of the conjugates described herein to treat diseases associated with activated macrophages, such as inflammatory diseases.

### Example 3: Scurfy Mouse Study

The scurfy mouse is a naturally occurring mouse model of the rare and fatal human disease, immunodysregulation polyendocrinopathy enteropathy X-linked syndrome (IPEX). It is an x-linked disorder that results in a functional defect in regulatory T cells (Tregs), which leads to lethal multi-organ inflammation. There is no known cure for this disease, although hematopoietic stem cell transplantation has been moderately effective in humans and in the scurfy mouse model. The study was designed to determine if macrophage targeting drug conjugates comprising a corticosteroid, specifically dexamethasone, can modulate the hyperactive autoimmune phenotype and reduce inflammation and/or organ damage sufficiently to extend life in the mice. When applied to humans, the conjugates may reduce inflammation to make hematopoietic stem cell transplantation less dangerous and more effective long term. In addition, the study was designed to determine if macrophage targeting steroid conjugates could show therapeutic effects comparable to the non-conjugated steroid, while limiting adverse effects.

The value of glucocorticoid therapy currently plays a pivotal role in many inflammatory diseases and still represents the most frequently used anti-inflammatory agent worldwide. The use of glucocorticoid therapy is restrained due to the associated significant adverse effects. The adverse effects relate to the fact that almost all cells in the body have glucocorticoid receptors. For anti-inflammatory use, the dose that is effective has numerous off target effects that limit the dose and duration of glucocorticoid use. The desired anti-inflammatory effect requires a systemic blood level sufficient to drive the glucocorticoid across the cell membrane in inflammatory cells so the glucocorticoid can bind the glucocorticoid receptor (GR) and create a complex that causes a genomic shift in the macrophage converting it from a pro-inflammatory phenotype (M1) to an anti-inflammatory phenotype (M2). An untargeted glucocorticoid at the same concentrations cause numerous genomic and non-genomic effects in most cells in the body resulting in unacceptable toxicity.

Male scurfy mice are divided into groups of 11-13 mice per group. Mice are studied from day 3 until maximal survival. The mice are evaluated for survival time. Untreated scurfy mice have a mean lifespan of 20 ±2 days.

Upon arrival the B6 females are caged (2 females per one male) with DBA1 male mice. The litters are genotyped with female carriers used for future breeding and male positives are then studied with the therapeutic test agent. The first litter is genotyped but not treated and animals were followed to establish control values. Future litter males are dosed subcutaneously, daily starting on day 3 with 1 mg of the test agent and followed daily to determine if the test agent improves quality of life, phenotype and longevity as compared to untreated controls.

The results may show that lifespan of scurfy mice can be extended using the conjugates described herein, as well as reducing symptoms associated with IPEX.

### Example 4: Effect of Macrophage Targeting Drug Conjugate on Blood Glucose

CC57BL/6 mice aged approximately 9 weeks were weighed and acclimated to facility conditions. Animals were assigned to 3 treatment groups based on their body weights, creating homogenous groups of 5 mice in each group. After fasting, blood glucose of levels of mice were tested at time 0, at which time the test drug was administered, then subsequently at 30, 60, 90 and 120 minutes post administration, using blood glucose test strips. The test drug administered to each group was dexamethasone 65 microgram (µg) per mouse to group 1; compound I5 at an amount of 100 µg per mouse to group 2; and compound I5 at an amount of 2500 µg per mouse to group 3.

The results were averaged and the changes from baseline were graphed and are shown in FIG. 3. As can be seen, dexamethasone administered a dosage of 65 mg (solid line) increases blood sugar levels in mice to levels of about 15% from at least 30 minutes to 120 minutes post administration.

When mice are administered 100 µg of compound I5 (dashed line) blood glucose is not increased above baseline throughout the period of 30 minutes to 120 minutes post-administration. This dosage, which corresponds to a molar equivalent of 65 µg of unconjugated dexamethasone, surprisingly did not cause elevations of blood glucose levels. When mice are administered 2500 µg each of compound I5 (dotted line), representing a 25-fold dosage of a molar equivalent of 65 µg of unconjugated dexamethasone, surprisingly no elevation of blood glucose level was seen relative to baseline.

These data indicate the potential of macrophage targeting drug conjugates, including, but not limited to I5, in treating inflammation, without raising blood glucose levels as seen with unconjugated glucocorticoids such as dexamethasone. There is potential for using such macrophage targeting drug conjugates for treatment of inflammatory disease in patients particularly sensitive to blood glucose elevation, such as diabetic patients or patients with metabolic syndrome.

### Example 5: Lipopolysaccharide (LPS) Model of Neuroinflammation

LPS is a cell-wall immunostimulatory component of gram-negative bacteria which was identified as a Toll-like receptor 4 (TLR-4) ligand. TLR-4 is expressed on microglia in the central nervous system, which once activated, produce proinflammatory cytokines, key mediators of the neuro-inflammatory process, including TNF-α, IL-1β, and prostaglandin E2. Administration of LPS to animals induces depression-like syndromes in animals and can be associated with neuroinflammatory diseases.

120 mice (female C57BL/6) mice approximately 6 weeks old were assigned into groups based on body weight, creating homogenous groups, and acclimated to facility conditions over 5 days.

A neuroinflammatory response was induced in all mice by intraperitoneal administration of 5 mg/kg of LPS. Test items were administered subcutaneously to the three groups as follows: Group 1: Compound I5, at an amount of 100 µg per mouse. Group 2: dexamethasone at 65 mg per mouse. Group 3: Vehicle. Test items were administered at the following time points: 30 minutes before LPS injection, 2 hours post-LPS injection and 10 hours post LPS injection (where applicable). 10 animals from each group were sacrificed at the following time points: 4, 8, 24 and 48 hours post LPS administration. Brains were collected and preserved in buffered 4% formaldehyde. Bronchoalveolar lavage fluid was extract from lungs of all animals.

Samples were analyzed for: IFN-gamma, IL-1b, IL-2, IL-4, IL-6, IL-10, MIP-1a, VEGF-A, TNFa, G-CSF, Eotaxin, GM-CSF, IL-1a, IL-3, IL-5, IL-7, IL-10, IL-12, IL-13, LIX, IL-15, IL-17, IP-10, KC, MCP-1, M-CSF, MIP-2, MIG, and RANTES.

The results were as follows:

Clinical score - Appearance - changes in mice appearance was seen 24 Hr. following LPS induction - the best clinical score was seen in group 1. Response to stimulus - changes in mice response were seen 24 hours following LPS induction - the best clinical score was seen in the group 1. Eye condition - changes in mice eye condition were seen in 24 hours following LPS induction - the best clinical score was seen in group 1. Respiration - changes in respiration were seen in 24 hours following LPS induction - the best clinical score was seen in the PIF-GC group. Total clinical score - Group 1 had the best clinical score compared to groups 2 and 3.

Cytokines levels in brains - statistical analysis revealed that treatment in group 1 had an immunomodulatory effect seen in IL-2 (4 and 8 Hr. post LPS), IL-10 (8 Hr. post LPS), VEGF a (8 and 24 Hr. post LPS) as compared to groups 2 and 3.

Cytokines levels in Bronchoalveolar lavage fluid- statistical analysis revealed that the group 1 treatment group had an immunomodulatory effect seen in IL-1 a and MCP1 (4 Hr. post LPS), MCP1 (8 Hr. post LPS). However, 24 Hr. post LPS induction group 2 had a more profound immunomodulatory effect seen in IL-1 a, IL-2 and MIP1 a as compared to groups 1 and 3.

Cytokines levels in serum - statistical analysis revealed that the group 1 treatment had an immunomodulatory effect seen in IL-17 and RANTES (4 Hr. post LPS), LIF (8 Hr. post LPS). However, 4 Hr. post LPS induction group 2 treatment had a more profound immunomodulatory effect seen in LIF, TNF a as compared to group 1 and group 3 treatment. Furthermore, 8 Hr. post LPS induction group 2 treatment had a more profound immunomodulatory effect seen in IL-17 as compared to group 1 and group 3, and 24 Hr. post LPS induction group 2 treatment had a more profound immunomodulatory effect seen in IL-1 b and RANTES.

The results indicated that the anti-inflammatory effects of compound I5 were seen in the CNS as evidenced by its difference, measured by CNS cytokine levels, from both untreated controls as well as an equimolar amount of free dexamethasone. This confirms that I5 was able to penetrate the blood-brain barrier and impact neuroinflammation in the brain. As I5 targets CD206 expressing macrophages in the CNS, the required efficiency of delivery of I5 across the BBB is less than free untargeted dexamethasone to achieve the desired macrophage dependent anti-inflammatory effects.

### Example 6: Penetration of the Blood-Brain Barrier in Vivo Using Macrophage Targeting Drug Conjugates

pharmacokinetic study was performed in mice to assess the systemic exposure of compound I5 in comparison to a reference steroid, dexamethasone, following a single intravenous (IV) injection to female ICR mice. Further, the excretion of compound I5 in urine, and the ability to cross the blood brain barrier, by analysis of brain cerebrospinal fluid (CSF), 1, 4, 8 and 24 hours post administration, were assessed for both test items.

Group 1 consisted of 6 mice and was divided into 2 subgroups of 3 mice. Mice of this group were administered a single IV injection of phosphate buffered saline, in an amount of 10 ml/kg.

Group 2 consisted of 24 mice and was divided into 8 subgroups of 3 mice. Mice of this group were administered a single IV injection of 200 mg/kg compound I5 in a solution of PBS and an amount of 10 ml/kg.

Group 3 consisted of 24 mice and was divided into 8 subgroups of 3 mice. Mice of this group were administered a single IV injection of 40 mg/kg dexamethasone sodium phosphate in a solution of PBS and an amount of 10 ml/kg.

For group 1, one subgroup had blood collected at 5 minutes post administration, and the other had blood collected 24 hours post administration. For groups 2 and 3, subgroups had blood collected at 5, 15, 30 minutes, 1, 2, 4, 8 and 24 hours post administration. The plasma pharmactokinetic results are detailed in the tables below. The analyte for group 2 was compound I5 and for group 3 was dexamethasone.

TABLE 2

PK parameters in plasma

| Group | Cₘₐₓ (ng/mL) | Tₘₐₓ (h) | AUCₗₐₛₜ (h^{∗}ng/mL) | Tₗₐₛₜ (h) | AUC_{INF} (h^{∗}ng/mL) | T_{½} (h) | Expᵣₑₗ ^{a} (%) |
|---|---|---|---|---|---|---|---|
| 2 | 44300 | 0.0833 | 18600 | 24.0 | 18700 | 4.23 | 4.2 |
| 3 | 27600 | 0.0833 | 88000 | 24.0 | 88000 | 1.83 | - |

a) Relative systemic exposure calculated as [AUCinf/dose (Compound I5)/AUCinf/dose (Dexamethasone)] x 100

TABLE 3

PK parameters in plasma, cont.

| Group | CL (mL/h/kg) | Vz (mL/kg) | Vss (mL/kg) | Cₘₐₓ/Dose (ng/mL/mg/kg) | | AUC_{INF}/Dose (h^{∗}ng/mL/mg/kg) |
|---|---|---|---|---|---|---|
| 2 | 10700 | 65300 | 12100 | 222 | 93.4 | |
| 3 | 455 | 1200 | 1300 | 689 | 2200 | |

TABLE 4

Parameters used for estimating t½ in plasma

| Group | Rsq | No points lambda z | Lambda z (1/h) | Lambda z lower (h) | Lambda z upper (h) | AUC_{%Extr} ₐₚ (%) | T_{½} (h) |
|---|---|---|---|---|---|---|---|
| 2 | 0.904 | 4 | 0.164 | 2 | 24 | 0.403 | 4.23 |
| 3 | 1.00 | 3 | 0.378 | 4 | 24 | 0.0134 | 1.83 |

The pooled concentrations vs time in urine is shown in Table 5:

TABLE 5

| Group | Nominal_time (h) | | | | |
|---|---|---|---|---|---|
| | 0.5 | 1.0 | 2.0 | 8.0 | 24.0 |
| | Concentration (ng/mL) | | | | |
| 2 | 119000 | 51000 | 23200 | 11400 | 7110 |
| 3 | 48900 | 56100 | 50500 | 27200 | 8370 |

The pooled concentrations vs time in brain (CSF) is shown in Table 6:

TABLE 6

| Group | Nominal_time (h) | | | |
|---|---|---|---|---|
| | 1 | 4 | 8 | 24 |
| | Concentration (ng/mL) | | | |
| 2 | 29.4 | 6.86 | BLQ | 10.5 |
| 3 | BLQ | BLQ | BLQ | BLQ |

BLQ represents levels below lowest level of quantification of 5.00 ng/mL

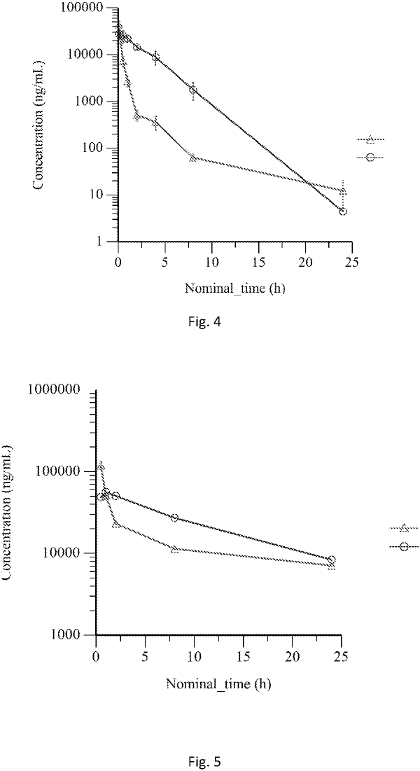

Mean plasma concentration versus time samples are presented in FIG. 4 (mean and SD values, n=3/time-point) for compound I5 (triangles) and dexamethasone (circles).

In the control group 1, no animal had been exposed to the test item, compound I5, as all samples were below LLOQ (5.00 ng/mL), whereas for Dexamethasone one animal (out of 6 animals) had about 6-fold levels above LLOQ (32.4 ng/mL).

All animals dosed with either Compound I5 or Dexamethasone were systemically exposed to the test items. However, three animals at the last time-point, 24 hr, had levels below LOQ, one animal in the Compound I5 group and two animals in the Dexamethasone group.

A two-phase elimination curve was seen for Compound I5 in plasma and the terminal elimination half-life was about 4.2 hours in mice after IV dosing. A one-phase elimination curve was seen for Dexamethasone in plasma and with a shorter half-life, 1.8 h.

The clearance for Compound I5 was higher than for Dexamethasone (10.7 vs. 0.455 L/h/kg). Further, the distribution volume, Vss, for Compound I5 was larger than for Dexamethasone (12.1 vs. 1.3 L/kg). Therefore, the dose-corrected Cₘₐₓ and AUC_{inf} were lower for Compound I5 than for Dexamethasone. The relative systemic exposure after administration of 200 mg Compound I5 was low, 4.2%, compared to 40 mg Dexamethasone.

Pooled urine concentration versus time profiles are shown for Compound I5 (triangles) and dexamethasone (circles) in FIG. 5.

In the control group, no animal had been exposed to the test item, Dexamethasone, as all samples were below LLOQ (5.00 ng/mL), whereas for Compound I5 the 24 h urine sample had levels above LLOQ (4010 ng/mL), approximately 50% lower than the 24 h urine sample in dose group 1, where 200 mg/kg was given (LLOQ, 5.00 ng/mL).

The pooled CSF concentration-time data listed in Table 6 indicate that Compound I5 may be able to penetrate the blood-brain barrier as levels from 6.86-29.4 ng/mL were detected (1.4-5.9-fold the LLOQ, 5.00 ng/mL). The highest level was seen at the 1^{st} time-point, 1 h post-dose. Dexamethasone did not penetrate the blood-brain barrier, or at least all collected samples were below LLOQ (5.00 ng/mL).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1-51. (canceled)

52. A compound having the formula M-L-D wherein M is a mannose moiety, L is a linker moiety and D is a drug moiety, wherein the drug moiety is a corticosteroid.

53. The compound according to claim 52 wherein L is a linker comprising a hydrazone moiety, an oxime moiety, an imine moiety, or a thioether moiety.

54. The compound according to claim 53 wherein L further comprises a carbonyl group.

55. The compound according to claim 54 wherein the carbonyl group is bound to the hydrazone moiety.

56. The compound according to claim 53 wherein L is a linker comprising an oxime moiety.

57. The compound according to claim 52 where L further comprises a C1-C6 alkyl group.

58. The compound according to claim 57 wherein the C1-C6 alkyl group is bound to the carbonyl group.

59. The compound according to claim 52 wherein the steroid is selected from the group consisting of: prednisone, dexamethasone, betamethasone, prednisolone, triamcinolone, hydrocortisone, fludrocortisone, and methylprednisolone.

60. The compound according to claim 59 wherein the drug is prednisone or dexamethasone.

61. A compound according to the formula: 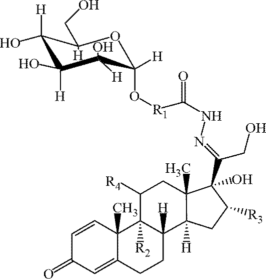
wherein R¹ is a direct bond; or C1-C12 straight alkyl or branched alkyl;
R² is hydrogen or fluorine;
R₃ is a hydrogen or methyl group.
R₄ is a hydroxyl group or a ketone group.

62. The compound according to claim 61 wherein R₁ is a direct bond.

63. The compound according to claim 61 wherein R₁ is a CH₂ group.

64. The compound according to claim 61 wherein R₁ is (methyl)ethyl group.

65. The compound according to claim 61 wherein R₁ is a pentyl group.

66. The compound according to claim 61 wherein R₂ and R₃ are H and R₄ is a ketone group.

67. The compound according to claim 61 wherein R₂ is fluorine, R₃ is methyl and R₄ is a hydroxyl group.

68. A compound according to the formula: 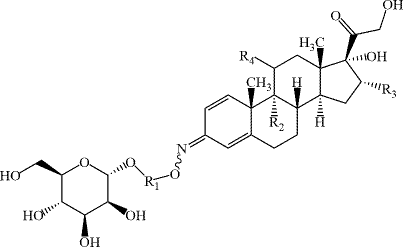
wherein R₁ is a direct bond; or C1-C12 straight alkyl or branched alkyl;
R₂ is hydrogen or fluorine;
R₃ is a hydrogen or methyl group; and
R₄ is a hydroxyl group or a ketone group.

69. The compound according to claim 68 wherein R₁ is a (CH₂)₃ group.

70. The compound according to claim 68 wherein R₂ and R₃ are H and R₄ is a ketone group.

71. The compound according to claim 68 wherein R₂ is fluorine, R₃ is methyl and R₄ is a hydroxyl group.
